(19) United States
(12) Patent Application Publication
(10) Pub. No.: US 2026/0306909 A1
(43) Pub. Date: Oct. 8, 2026

(54) ELECTRONIC APPARATUS FOR PROVIDING EXERCISE ROUTINE AND CONTROLLING METHOD THEREOF

(71) Applicant: SAMSUNG ELECTRONICS CO., LTD., Suwon-si (KR)

(72) Inventors: Jieung JUN, Suwon-si (KR); Chungdo KIM, Suwon-si (KR); Minkyung HWANG, Suwon-si (KR)

(21) Appl. No.: 19/224,633

(22) Filed: May 30, 2025

Related U.S. Application Data
(63) Continuation of application No. PCT/KR2025/004292, filed on Apr. 1, 2025.

(30) Foreign Application Priority Data
Jun. 21, 2024 (KR) 10-2024-0081309
Jul. 9, 2024 (KR) 10-2024-0090452

(51) Int. Cl. A63B 24/00

(52) U.S. Cl. CPC A63B 24/0075; A63B 24/0062; A63B 2024/0065; A63B 2230/705

(57) ABSTRACT

An electronic apparatus includes a sensor, a display, memory storing instructions, and at least one processor, and the instructions, when executed by the at least one processor, cause the electronic apparatus to obtain data about a user, obtain information about an exercise routine corresponding to the user based on the obtained data, obtain information about the user's exercise performance ability while the user is exercising using the exercise routine, and update the exercise routine based on the exercise performance ability.

### CROSS-REFERENCE TO RELATED APPLICATION

This application is a continuation application of International Application No. PCT/KR2025/004292 designating the United States, filed on Apr. 1, 2025, in the Korean Intellectual Property Receiving Office and claiming priority to Korean Patent Application No. 10-2024-0081309 filed on Jun. 21, 2024 and to Korean Patent Application No. 10-2024-0090452 filed on Jul. 9, 2024 in the Korean Intellectual Property Office, the disclosure of which is incorporated by reference herein in its entirety.

### BACKGROUND

### (1) Field

The present disclosure relates to an electronic apparatus and a controlling method thereof, and more particularly to, an electronic apparatus capable of providing and updating an exercise routine based on data about a user and a controlling method thereof.

### (2) Description of the Related Art

In recent years, applications and programs that manage the user's health using various electronic apparatuses such as smart phones and smart watches have been developed. In particular, recently, programs that set exercise routines, which are combinations of various exercises, as a goal and manage the user's exercise according to the set exercise routines have been provided.

The above-described information may be provided as background art for the purpose of helping to understand the present disclosure. No claim or determination is made as to whether any of the above is applicable as prior art related to the present disclosure.

### SUMMARY

An electronic apparatus, according to an embodiment, includes a display, memory storing instructions, and at least one processor, and the instructions, when executed by the at least one processor, cause the electronic apparatus to obtain data about a user, obtain information about an exercise routine corresponding to the user based on the obtained data, obtain information about an exercise performance ability of the user while the user is exercising using the exercise routine, and update the exercise routine based on the exercise performance ability.

The instructions, when executed by the at least one processor, may cause the electronic apparatus to provide query information to obtain information about at least one of the user's current condition or exercise ability, and obtain data about the user according to a user input entered based on the query information.

The query information may include at least one of query information to inquire about the user's diet, sleeping, drinking alcohol, medication, activity amount, body fat, target exercise area, or satisfaction about an existing exercise routine.

The instructions, when executed by the at least one processor, may cause the electronic apparatus to obtain information about whether the user is able to perform exercise and information about exercise intensity based on the obtained data, identify an exercise goal based on the information about whether the user is able to perform exercise and information about exercise intensity, analyze feedback information about the existing exercise routine, and obtain information about an exercise routine corresponding to the user based on the exercise goal and the feedback information.

The instructions, when executed by the at least one processor, may cause the electronic apparatus to, based on the user drinking alcohol within a preset drinking period, taking a preset drug within a preset drug period, eating less than a threshold amount of food, sleeping less than a threshold sleeping time, or having a vitality score less than a preset vitality value, obtain information about an exercise routine of first intensity, based on the user not drinking alcohol within the preset drinking period, not taking the preset drug within the preset drug period, eating more than the threshold amount of food, sleeping more than the threshold sleeping time, or having the vitality score more than the preset vitality value, obtain information about an increase in body fat, based on the increase in body fat being less than a preset body fat value, obtain information about an exercise routine of second intensity, based on the increase in body fat being more than the preset body fat value, obtain information about an exercise routine of third intensity, and the first intensity may be lower than the second intensity, and the second intensity may be lower than the third intensity.

The instructions, when executed by the at least one processor, may cause the electronic apparatus to calculate a condition score based on information about the user's meal amount, the user's last meal time, the user's sleep amount, whether the user drinks alcohol, the user's activity amount, the user's change in body fat, and a degree of performance of the existing exercise routine, and obtain information about an exercise routine corresponding to the user by adjusting an exercise goal of the existing exercise routine based on the existing exercise routine and the condition score.

The instructions, when executed by the at least one processor, may cause the electronic apparatus to obtain information about the user's condition and information about a progress of the exercise routine while the user is exercising using the exercise routine, and update detailed settings of a next exercise included in the exercise routine based on the information about the user's condition and the information about the progress of the exercise routine.

The exercise routine may be configured to include a plurality of exercises and a rest period between the plurality of exercises, and the instructions, when executed by the at least one processor, may cause the electronic apparatus to provide information about the updated detailed settings of a next exercise during the rest period.

The detailed settings of the exercise may be configured to include at least one of the number of progressions of exercise, a weight of equipment required for exercise, or the number of sets of exercise.

The exercise routine may be configured to include a warm-up exercise, a main exercise, a rest period, and a cool-down exercise.

A controlling method of an electronic apparatus, according to an embodiment, includes obtaining data about a user, obtaining information about an exercise routine corresponding to the user based on the obtained data, obtaining information about an exercise performance ability of the user while the user is exercising using the exercise routine, and updating the exercise routine based on the exercise performance ability.

The obtaining data about a user may include providing query information to obtain information about at least one of the user's current condition or exercise ability, and obtaining data about the user according to a user input entered based on the query information.

The query information may include at least one of query information to inquire about the user's diet, sleeping, drinking alcohol, medication, activity amount, body fat, target exercise area, or satisfaction about an existing exercise routine.

The obtaining information about an exercise routine may include obtaining information about whether the user is able to perform exercise and information about exercise intensity based on the obtained data, identifying an exercise goal based on the information about whether the user is able to perform exercise and information about exercise intensity, analyzing feedback information about the existing exercise routine, and obtaining information about an exercise routine corresponding to the user based on the exercise goal and the feedback information.

The obtaining information about an exercise routine may include, based on the user drinking alcohol within a preset drinking period, taking a preset drug within a preset drug period, eating less than a threshold amount of food, sleeping less than a threshold sleeping time, or having a vitality score less than a preset vitality value, obtaining information about an exercise routine of first intensity, based on the user not drinking alcohol within the preset drinking period, not taking the preset drug within the preset drug period, eating more than the threshold amount of food, sleeping more than the threshold sleeping time, or having the vitality score more than the preset vitality value, obtaining information about an increase in body fat, based on the increase in body fat being less than a preset body fat value, obtaining information about an exercise routine of second intensity, based on the increase in body fat being more than the preset body fat value, obtaining information about an exercise routine of third intensity, and the first intensity may be lower than the second intensity, and the second intensity may be lower than the third intensity.

The obtaining information about an exercise routine may include calculating a condition score based on information about the user's meal amount, the user's last meal time, the user's sleep amount, whether the user drinks alcohol, the user's activity amount, the user's change in body fat, and a degree of performance of the existing exercise routine, and obtaining information about an exercise routine corresponding to the user by adjusting an exercise goal of the existing exercise routine based on the existing exercise routine and the condition score.

The updating may include obtaining information about the user's condition and information about a progress of the exercise routine while the user is exercising using the exercise routine, and updating detailed settings of a next exercise included in the exercise routine based on the information about the user's condition and the information about the progress of the exercise routine.

The exercise routine may be configured to include a plurality of exercises and a rest period between the plurality of exercises, and the updating may include providing information about the updated detailed settings of a next exercise during the rest period.

The detailed settings of the exercise may be configured to include at least one of the number of progressions of exercise, a weight of equipment required for exercise, or the number of sets of exercise.

The exercise routine may be configured to include a warm-up exercise, a main exercise, a rest period, and a cool-down exercise.

A computer program product, according to an embodiment, includes a storage medium storing instructions configured to be executed by at least one processor of an electronic apparatus to perform a plurality of operations. The operations include obtaining data about a user, obtaining information about an exercise routine corresponding to the user based on the obtained data, obtaining information about an exercise performance ability of the user while the user is exercising using the exercise routine, and updating the exercise routine based on the exercise performance ability.

### BRIEF DESCRIPTION OF THE DRAWINGS

With respect to the description in the drawings, the same or similar reference numerals may be used for the same or similar components.

FIG. 1 is a block diagram of an electronic apparatus according to an embodiment;

FIG. 2A is a perspective view of a front side of an electronic apparatus according to an embodiment;

FIG. 2B is a perspective view of a rear side of an electronic apparatus according to an embodiment;

FIG. 2C is an exploded perspective view of an electronic apparatus according to an embodiment;

FIG. 3 is a view illustrating a system that includes an electronic apparatus and an external device according to an embodiment;

FIG. 4 is a block diagram illustrating configuration of an electronic apparatus according to an embodiment;

FIGS. 5A and 5B are views illustrating an application included in an electronic apparatus according to an embodiment;

FIGS. 6A, 6B, 6C, and 6D are views provided to explain a method of generating an exercise or an exercise routine using an electronic apparatus according to an embodiment;

FIGS. 7A, 7B, and 7C are views provided to explain a method of generating an exercise or an exercise routine using an external device according to an embodiment;

FIG. 8 is a flowchart provided to explain a method of providing and updating an exercise routine based on data about a user according to an embodiment;

FIG. 9 depicts views illustrating query items for obtaining data about a user according to an embodiment;

FIG. 10 is a flowchart provided to explain a method of creating an exercise routine based on data about a user according to an embodiment;

FIG. 11 is a flowchart provided to specifically explain a method of creating an exercise routine based on data about a user according to an embodiment;

FIG. 12 is a view provided to explain a method of calculating a user's health score according to an embodiment;

FIG. 13 depicts views illustrating information about an exercise routine provided while performing an exercise routine according to an embodiment; and

FIG. 14 are views illustrating a process of updating an exercise routine based on a user's exercise ability, according to an embodiment.

### DETAILED DESCRIPTION

The embodiments of the present disclosure may be modified in various ways, and may have various embodiments, so specific embodiments are illustrated in the drawings and described in detail in the detailed description. However, it is to be understood that the disclosure is not limited to specific exemplary embodiments, but include all modifications, equivalents, and/or alternatives according to exemplary embodiments of the disclosure. Throughout the description of the accompanying drawings, similar components may be denoted by similar reference numerals.

In describing the disclosure, when it is decided that a detailed description for the known functions or configurations related to the disclosure may unnecessarily obscure the gist of the disclosure, the detailed description therefor will be omitted.

In addition, the following exemplary embodiments may be modified in several different forms, and the scope of the technical spirit of the disclosure is not limited to the following exemplary embodiments. Rather, these exemplary embodiments make the disclosure thorough and complete, and are provided to completely transfer the spirit of the disclosure to those skilled in the art.

Terms used in the disclosure are used only to describe specific exemplary embodiments rather than limiting the scope of the disclosure. Singular forms are intended to include plural forms unless the context clearly indicates otherwise. For example, as used herein, “a”, “an,” “the,” and “at least one” do not denote a limitation of quantity, and are intended to include both the singular and plural, unless the context clearly indicates otherwise. Thus, reference to “an” element in a claim followed by reference to “the” element is inclusive of one element and a plurality of the elements. For example, “an element” has the same meaning as “at least one element,” unless the context clearly indicates otherwise.

In the disclosure, the expressions “have”, “may have”, “include” or “may include” used herein indicate existence of corresponding features (e.g., elements such as numeric values, functions, operations, or components), but do not exclude presence of additional features.

In the disclosure, the expressions “A or B”, “at least one of A or/and B”, or “one or more of A or/and B”, and the like may include any and all combinations of one or more of the items listed together. For example, the term “A or B”, “at least one of A and B”, or “at least one of A or B” may refer to all of the case (1) where at least one A is included, the case (2) where at least one B is included, or the case (3) where both of at least one A and at least one B are included.

Expressions “first”, “second”, “1st,” “2nd,” or the like, used in the disclosure may indicate various components regardless of sequence and/or importance of the components, will be used only in order to distinguish one component from the other components, and do not limit the corresponding components.

When it is described that an element (e.g., a first element) is referred to as being “(operatively or communicatively) coupled with/to” or “connected to” another element (e.g., a second element), it should be understood that it may be directly coupled with/to or connected to the other element, or they may be coupled with/to or connected to each other through an intervening element (e.g., a third element).

On the other hand, when an element (e.g., a first element) is referred to as being “directly coupled with/to” or “directly connected to” another element (e.g., a second element), it should be understood that there is no intervening element (e.g., a third element) in-between. Further, the description in the disclosure that one element is “on top of” another element not only includes a case where the one element contacts the another element, but also a case where still another element exists between the two elements. For example, when an element is referred to as being related to another element such as being “on” another element, it can be directly on the other element or intervening elements may be present therebetween. In contrast, when an element is referred to as being related to another element such as being “directly on” another element, there are no intervening elements present.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

An expression “~configured (or set) to” used in the disclosure may be replaced by an expression, for example, “suitable for,” “having the capacity to,” “~designed to,” “~adapted to,” “~made to,” or “~capable of” depending on a situation. A term “~configured (or set) to” may not necessarily mean “specifically designed to” in hardware.

Instead, an expression “~an apparatus configured to” may mean that an apparatus “is capable of” together with other apparatuses or components. For example, a “processor configured (or set) to perform A, B, and C” may mean a dedicated processor (e.g., an embedded processor) for performing the corresponding operations or a generic-purpose processor (e.g., a central processing unit (CPU) or an application processor) that may perform the corresponding operations by executing one or more software programs stored in a memory device.

In exemplary embodiments, a ‘module’ or a ‘unit’ may perform at least one function or operation, and be implemented as hardware or software or be implemented as a combination of hardware and software. In addition, a plurality of ‘modules’ or a plurality of ‘unit’ may be integrated into at least one module and be implemented as at least one processor except for a ‘module’ or a ‘unit’ that needs to be implemented as specific hardware.

Meanwhile, various elements and areas in the drawings are schematically drawn in the drawings. Therefore, the technical concept of the disclosure is not limited by a relative size or spacing drawn in the accompanying drawings.

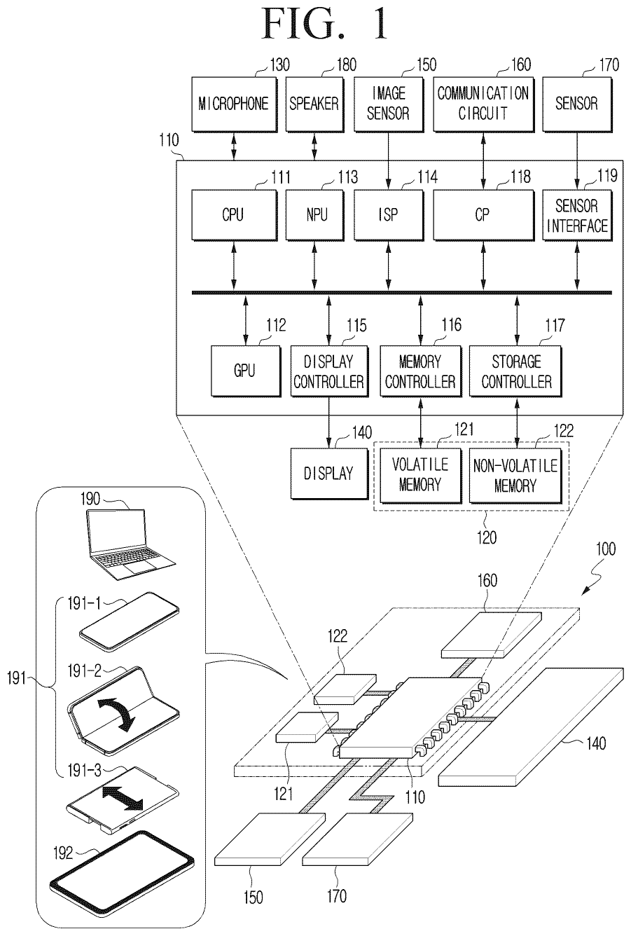

FIG. 1 is a block diagram of an electronic apparatus according to an embodiment.

FIG. 1 is a block diagram of an exemplary electronic apparatus capable of performing operations described according to an embodiment.

Referring to FIG. 1, an electronic apparatus 100 may be implemented in various forms that can be worn by a user, such as smart watch, smart band, smart ring, wireless earphones, or smart glasses. The components, their relationships, and their functions illustrated in FIG. 1 are exemplary only, and do not limit the implementations described or claimed in this document. The electronic apparatus 100 may be referred to as a mobile device, a user device, a multi-function device, a portable device, or a server.

The electronic apparatus 100 may include components including at least one processor 110, at least one memory 120 (hereinafter, referred to as memory 120), at least one display 140 (hereinafter, referred to as a display 140), at least one image sensor 150 (hereinafter, referred to as an image sensor 150), at least one communication circuitry 160 (hereinafter, referred to as a communication circuitry 160), and/or at least one sensor 170 (hereinafter, referred to as a sensor 170). The above components are exemplary only. For example, the electronic apparatus 100 may include other components (e.g., power management integrated circuitry (PMIC), audio processing circuitry, antennas, rechargeable batteries, or input/output interfaces). For example, some components may be omitted from the electronic apparatus 100. For example, some components may be integrated into a single component.

The at least one processor 110 may be implemented as one or more integrated circuit (IC) (or circuitry)) chips, and may execute various data processing. The at least one processor 110 may include at least one electrical circuit, and may individually or collectively distribute and process instructions (or programs, data) stored in the memory 120. The at least one processor 110 may include a processor assembly including one or more processing circuitry. The at least one processor 110 may include any processing circuitry that is operative to control the performance and operation of one or more components of the electronic apparatus 100 (e.g., memory 120, microphone 130, display 140, image sensor 150, communication circuitry 160, sensor 170, and/or speakers 180). For example, the at least one processor 110 (e.g., application processor (AP)) may be implemented as a system on chip (SoC) (e.g., a single chip or a chipset). For example, the at least one processor 110 may be implemented as multiple cores (or at least one core circuit), multiple chips, or multiple chipsets. For example, the at least one processor 110 may include one or more processing circuitry. For example, the at least one processor 110 may include one or more processing circuitry configured to individually and/or collectively perform various functions of the present disclosure. As a non-limiting example, at least a portion of the at least one processor 110 may be included in a first chip of the electronic apparatus 100, and at least another portion of the at least one processor 110 may be included in a second chip of the electronic apparatus 100, which is different from the first chip of the electronic apparatus 100.

For example, the at least one processor 110 may include a central processing unit (CPU) 111, a graphics processing unit (GPU) 112, a neural processing unit (NPU) 113, an image signal processor (ISP) 114, a display controller 115, a memory controller 116, a storage controller 117, a communication processor (CP) 118, and/or a sensor interface 119. These components of the at least one processor 110 are exemplary only. For example, the at least one processor 110 may further include other components. For example, some components of the at least one processor 110 may be omitted from the at least one processor 110. For example, some components of the at least one processor 110 may be included as separate components of the electronic apparatus 100 outside of the at least one processor 110. For example, some components of the at least one processor 110 (e.g., the memory controller 116) may be included within other components (e.g., at least a portion of the memory 120, an interface (e.g., available for connection to at least one component of the electronic apparatus 100, the display 140, and/or the image sensor 150).

The at least one processor 110 may cause other components of the electronic apparatus 100 to perform various operations by executing instructions stored in the memory 120. The CPU 111 (or central processing circuitry) may be configured to control components of the at least one processor 110 based on execution of instructions stored in the memory 120 (e.g., volatile memory 121 and/or non-volatile memory 122). The GPU 112 (or graphics processing circuitry) may be configured to execute parallel computations (e.g., rendering). The NPU 113 (or neural processing circuitry or artificial intelligence (AI) chip) may be configured to execute computations for an AI model (e.g., convolution computation). The ISP 114 (or image signal processing circuitry) may be configured to process raw images obtained via the image sensor 150 into a format suitable for components within the electronic apparatus 100 or components of the at least one processor 110. The display controller 115 (or display control circuitry or display processing unit (DPU)) may be configured to process images obtained from the CPU 111, the GPU 112, the ISP 114, or the memory 120 (e.g., volatile memory 121) into a format suitable for the display 140. The memory controller 116 (or memory control circuitry) may be configured to control reading data from volatile memory 121 and writing data to the volatile memory 121. The storage controller 117 (or storage control circuitry) may be configured to control reading data from the non-volatile memory 122 and writing data to the non-volatile memory 122. The CP 118 (communication processing circuitry) may be configured to process data obtained from a component of the at least one processor 110 into a format suitable for transmission via the communication circuitry 160 to another electronic apparatus, or to process data obtained via the communication circuitry 160 from another electronic apparatus into a format suitable for processing of a component of the at least one processor 110. For example, the communication circuitry 160 may include one or more communication circuits. The sensor interface 119 (or sensing data processing circuitry or sensor hub) may be configured to process data about a state of the electronic apparatus 100 and/or a state of the environment surrounding the electronic apparatus 100, obtained via the sensor 170, into a format suitable for a component of the at least one processor 110.

The memory 120 may include one or more storage media (or one or more storage devices). For example, the memory 120 may include a memory assembly including one or more storage media. For example, the one or more storage media may include a hard drive, flash memory, permanent memory such as read-only memory (ROM) (e.g., non-volatile memory 122), semi-permanent memory such as random access memory (RAM) (e.g., volatile memory 121), any other suitable type of storage (or storage assembly), or any combination thereof. The memory 120 may include cache memory, which is one or more different types of memory used to temporarily store data for a function or feature of the electronic apparatus 100. As a non-limiting example, the cache memory may be included within at least one processor 110. The memory 120 may be fixedly embedded within the electronic apparatus 100, or may be incorporated into one or more suitable types of components (e.g., subscriber identity module (SIM) card and/or secure digital (SD) card) that may be repeatedly inserted into and removed from the electronic apparatus 100.

For example, the memory 120 may store one or more software applications, such as operating system (or system) software application, firmware software application, driver software application, plug-in (e.g., add-in, add-on, and/or applet) software application, and/or any other suitable software application. For example, the one or more software applications may include instructions executable by the at least one processor 110. For example, the memory 120 may store instructions that that can be called by an application programming interface (API). For example, the memory 120 may store instructions within a library.

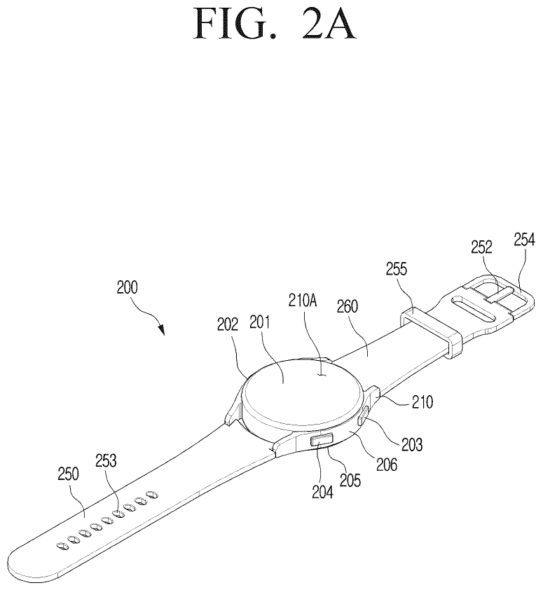

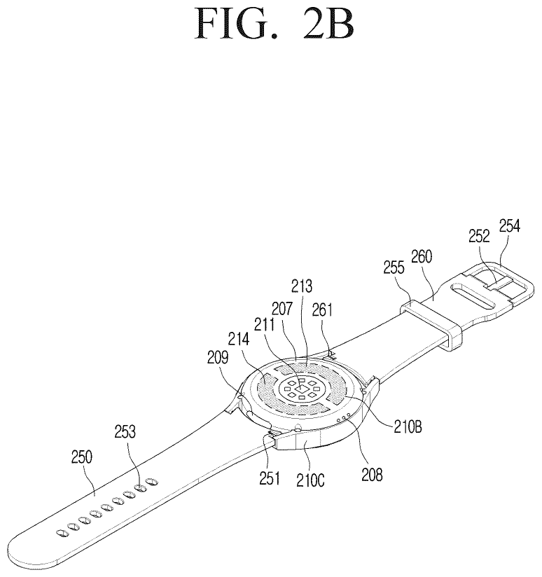

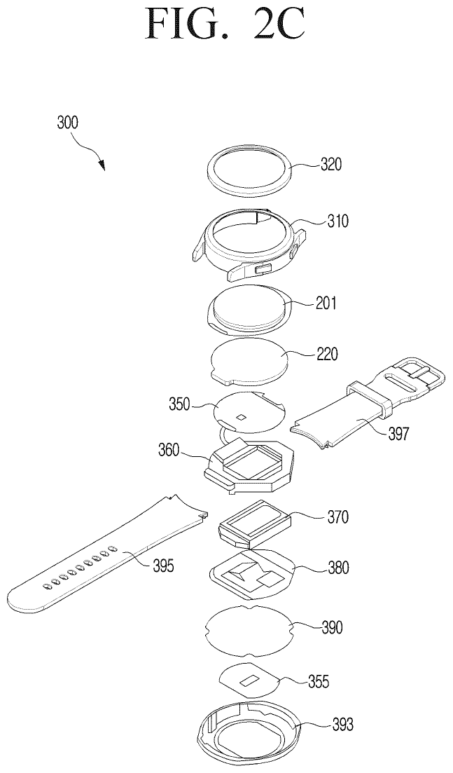

FIG. 2A is a perspective view of a front side of an electronic apparatus according to an embodiment, and FIG. 2B is a perspective view of a rear side of an electronic apparatus according to an embodiment.

Referring to FIGS. 2A and 2B, an electronic apparatus 200 (e.g., an electronic apparatus 101 of FIG. 1) according to an embodiment may include a housing 210 including a first side (or front side) 210A, a second side (or back side) 210B, and a side surface 210C surrounding a space between the first side 210A and the second side 210B, and fastening members 250, 260 connected to at least a portion of the housing 210 and configured to detachably fasten the electronic apparatus 200 to a body part (e.g., wrist or ankle) of a user. In other embodiment (not shown), the housing may refer to a structure forming a portion of the first side 210A, the second side 210B, and the side surface 210C of FIGS. 2A and 2B. According to an embodiment, the first side 210A may be formed by a front plate 201 that is at least partially substantially transparent (e.g., a glass plate including various coating layers, or a polymer plate). The second side 210B may be formed by a substantially opaque back plate 207. The back plate 207 may be formed by, for example, a coated or colored glass, a ceramic, a polymer, a metal (e.g., aluminum, stainless steel (STS), or magnesium), or a combination of at least two of the foregoing materials. The side surface 210C above is coupled with the front plate 201 and the back plate 207, and may be formed by a side bezel structure (or “side member”) 206 including metal and/or polymer. In some embodiments, the back plate 207 and the side bezel structure 206 may be formed integrally, and may include the same material (e.g., a metal material such as aluminum). The fastening members 250, 260 may be formed of various materials and shapes. The integral and multiple unit links may be formed to be mutually movable by a woven material, leather, rubber, urethane, metal, ceramic, or a combination of at least two of the above materials.

According to an embodiment, the electronic apparatus 200 may include at least one of a display 220 (see FIG. 2C), audio modules 205, 208, a sensor module 211, key input devices 202, 203, 204, and a connector hole 209. In some embodiments, the electronic apparatus 200 may omit at least one of the components (e.g., the key input devices 202, 203, 204, the connector hole 209, or the sensor module 211) or may additionally include other components.

The display 220 may be visually exposed, for example, through a significant portion of the front plate 201. The shape of the display 220 may correspond to the shape of the front plate 201, and may have various shapes such as a circle, an oval, or a polygon. The display 220 may be combined with or placed adjacent to a touch detection circuit, a pressure sensor capable of measuring the intensity (pressure) of a touch, and/or a fingerprint sensor.

The audio modules 205, 208 may include a microphone hole 205 and a speaker hole 208. The microphone hole 205 may have a microphone (e.g., the microphone 130 of FIG. 1) placed inside to obtain external sound, and in some embodiments, a plurality of microphones may be placed to detect the direction of sound. The speaker hole 208 may be used as an external speaker and a receiver for calls. In some embodiments, the speaker hole 208 and the microphone hole 205 may be implemented as one hole, or a speaker (e.g., the speaker 180 of FIG. 1) may be included without the speaker hole 208 (e.g., piezo speaker).

The sensor 211 (e.g., the sensor 170 of FIG. 1) may generate an electrical signal or a data value corresponding to an internal operating state of the electronic apparatus 200 or an external environmental state. The sensor 211 may include, for example, a biometric sensor 211 (e.g., heart rate monitor sensor) disposed on the second side 210B of the housing 210. The electronic apparatus 200 may further include at least one of a non-illustrated sensor, for example, a gesture sensor, a gyro sensor, a barometric pressure sensor, a magnetic sensor, an acceleration sensor, a grip sensor, a color sensor, an IR (infrared) sensor, a biometric sensor, a temperature sensor, a humidity sensor, or an illuminance sensor.

The sensor 211 may include electrode regions 213, 214 forming a part of the surface of the electronic apparatus 200 and a bio-signal detection circuit (not shown) electrically connected to the electrode region 213, 214. For example, the electrode region 213, 214 may include the first electrode region 213 and the second electrode region 214 disposed on the second surface 210B of the housing 210. The sensor 211 may be configured such that the electrode regions 213, 214 obtain an electrical signal from a part of the user's body, and the bio-signal detection circuit detects the user's bio-information based on the electrical signal.

The key input devices 202, 203, 204 may include a wheel key 202 disposed on the first side 210A of the housing 210 and rotatable in at least one direction, and/or side key buttons 203, 204 disposed on the side surface 210C of the housing 210. The wheel key may have a shape corresponding to the shape of the front plate 201. In another embodiment, the electronic apparatus 200 may not include some or all of the above-mentioned key input devices 202, 203, 204, and the key input devices 202, 203, 204 that are not included may be implemented in another shape, such as a soft key, on the display 220. The connector hole 209 may include another connector hole (not shown) that may accommodate a connector (e.g., a USB connector) for transmitting and receiving power and/or data to and from an external device, and may accommodate a connector for transmitting and receiving audio signals to and from an external device. The electronic apparatus 200 may further include, for example, a connector cover (not shown) that covers at least a portion of the connector hole 209 and blocks the inflow of external foreign substances into the connector hole.

The fastening members 250, 260 may be detachably fastened to at least a portion of the housing 210 using locking members 251, 261. The fastening members 250, 260 may include one or more of a fixing member 252, a fixing member fastening hole 253, a band guide member 254, and a band fixing ring 255.

The fixing member 252 may be configured to fix the housing 210 and the fastening members 250, 260 to a portion of the user's body, such as a wrist or an ankle. The fixing member fastening hole 253 may correspond to the fixing member 252 to fix the housing 210 and the fastening members 250, 260 to a portion of the user's body. The band guide member 254 may be configured to limit the range of movement of the fixing member 252 when the fixing member 252 is fastened to the fixing member fastening hole 253, thereby allowing the fastening member 250, 260 to be fastened in close contact with a part of the user's body. The band fixing ring 255 may limit the range of movement of the fastening members 250, 260 while the fixing member 252 and the fixing member fastening hole 253 are fastened.

FIG. 2C is an exploded perspective view of an electronic apparatus according to an embodiment.

Referring to FIG. 2C, an electronic apparatus 300 (e.g., the electronic apparatus 101 of FIG. 1 or the electronic apparatus 200 of FIGS. 2A and 2B) includes a side bezel structure 310, a wheel key 320 (e.g., the wheel key 202 of FIGS. 2A and 2B), the front plate 201, the display 220, a first antenna 350, a second antenna 355, a support member 360 (e.g., bracket), a battery 370, a printed circuit board 380, a sealing member 390, a back plate 393 (e.g., the back plate 207 of FIGS. 2A and 2B), and fastening members 395, 397 (e.g., the fastening members 250, 260 of FIGS. 2A and 2B). At least one of the components of the electronic apparatus 300 may be the same as, or similar to, at least one of the components of the electronic apparatus 100 of FIG. 1, or at least one of the components of the electronic apparatus 200 of FIGS. 2A and 2B, and redundant descriptions will be omitted below. The support member 360 may be disposed within the electronic apparatus 300 and may be connected to the side bezel structure 310, or may be integrally formed with the side bezel structure 310. The support member 360 may be formed of, for example, a metallic material and/or a non-metallic (e.g., polymer) material. The printed circuit board 380 may be equipped with a processor (e.g., the processor 110 of FIG. 1), memory (e.g., the memory 120 of FIG. 1), and/or an interface. The processor may include, for example, one or more of a central processing unit, a graphic processing unit (GPU), an application processor, a sensor processor, or a communication processor.

The memory may include, for example, volatile memory (e.g., the volatile memory 121 of FIG. 1) or non-volatile memory (e.g., the non-volatile memory 122 of FIG. 1). The interface may include, for example, a high definition multimedia interface (HDMI), a universal serial bus (USB) interface, an SD card interface, and/or an audio interface. The interface may, for example, electrically or physically connect the electronic apparatus 300 to an external device, and may include a USB connector, an SD card/MMC connector, or an audio connector.

The battery 370 is a device for powering at least one component of the electronic apparatus 300, and may include, for example, a non-rechargeable primary cell, a rechargeable secondary cell, or a fuel cell. At least a portion of the battery 370 may be disposed substantially on the same plane as, for example, the printed circuit board 380. The battery 370 may be disposed integrally within the electronic apparatus 300, or may be disposed detachably from the electronic apparatus 300.

The first antenna 350 may be disposed between the display 220 and the support member 360. The first antenna 350 may include, for example, a near field communication (NFC) antenna, a wireless charging antenna, and/or a magnetic secure transmission (MST) antenna. The first antenna 350 may, for example, perform short-range communication with an external device, wirelessly transmit and receive power required for charging, and transmit a magnetic-based signal including a short-range communication signal or payment data. In another embodiment, the antenna structure may be formed by a part or combination of the side bezel structure 310 and/or the support member 360.

The second antenna 355 may be disposed between the printed circuit board 380 and the back plate 393. The second antenna 355 may include, for example, a near field communication (NFC) antenna, a wireless charging antenna, and/or a magnetic secure transmission (MST) antenna. The second antenna 355 may, for example, perform short-range communication with an external device, wirelessly transmit and receive power required for charging, and transmit a magnetic-based signal including a short-range communication signal or payment data. In another embodiment, the antenna structure may be formed by a part or combination of the side bezel structure 310 and/or the back plate 393.

The sealing member 390 may be located between the side bezel structure 310 and the back plate 393. The sealing member 390 may be configured to block moisture and foreign substances from entering the space surrounded by the side bezel structure 310 and the back plate 393 from the outside.

Meanwhile, the electronic apparatus may provide an exercise routine. A prior art method of providing an exercise routine includes performing exercises of predefined types and intensities for a preset period of time. Such an exercise routine provided a uniform exercise program since it did not consider the user's physical strength, weight, or condition. In addition, the prior art method of providing an exercise routine did not reflect the user's feedback, and there is no continuous management and investigation, so it is questionable whether the proposed program is actually effective.

To address these issues, according to an embodiment, the electronic apparatus 100 may obtain user data to create an exercise routine, and may update the exercise routine in real time.

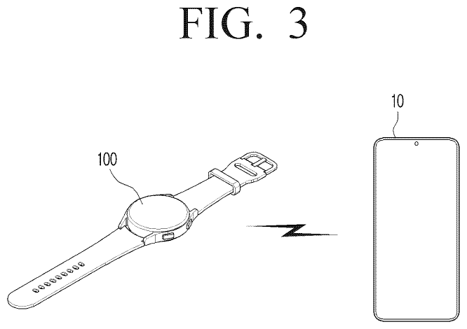

FIG. 3 is a view illustrating a system that includes an electronic apparatus and an external device according to an embodiment. As shown in FIG. 3, the system may include the electronic apparatus 100 and an external device 10. Here, the electronic apparatus 100 may be implemented as a wearable device that can be worn by the user, such as smart watch, smart ring, smart glasses, or head mounted display (HMD) device, but this is only one embodiment and may be implemented as a device such as another user terminal (e.g., a smartphone). The external device 10 may be a smartphone, as shown in FIG. 3, but this is only one embodiment and may be implemented as a variety of user terminals, such as tablet personal computer (PC), laptop PC, or desktop PC.

The electronic apparatus 100 may obtain data about the user. The data about the user (hereinafter, referred to as “user data”) is data to determine the user's current physical condition and exercise ability, and may include data about the user's profile (gender, age), data about the user's physical information, data about the user's condition, data about the user's exercise experience, data about the user's injury history, data about the user's physical strength, and data about the user's preferred type of exercise.

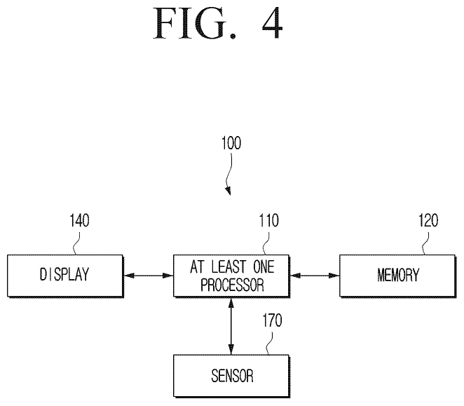

In one or more embodiments, the electronic apparatus 100 may obtain the user data using a sensor included in the electronic apparatus 100 (e.g., the sensor 170 in FIG. 4). Further, the electronic apparatus 100 may provide the user with query information to obtain information about at least one of the user's current condition and exercise ability. The electronic apparatus 100 may also obtain the user data in response to a user input entered based on the query information.

In one or more embodiments, the electronic apparatus 100 may obtain the user data from the external device 10. Here, the external device 10 may also obtain the user data through a sensor or obtain the user data using the query information. Subsequently, the external device 10 may transmit the obtained user data to the electronic apparatus 100.

The electronic apparatus 100 may obtain information about an exercise routine corresponding to the user based on the obtained data. The exercise routine is an exercise plan to be performed regularly over a preset period of time, and may include a plurality of exercises and rest periods. Further, the plurality of exercises may include warm-up, main exercise, and cool-down. Here, the exercise routine may be called by various terms such as exercise program, exercise schedule, fitness plan, training plan, exercise set, fitness routine, and training routine. In addition, the information about an exercise routine may include information about exercise goals, information about exercise types, information about exercise time and frequency, and information about exercise intensity.

In one or more embodiments, the electronic apparatus 100 may receive information about an exercise routine obtained based on the user data from the external device 10.

The electronic apparatus 100 may use the exercise routine to obtain information about the user's exercise performance ability while the user is performing the exercise. The user's exercise performance ability may include whether the user has achieved the goals of the exercise routine and the user's level of fatigue.

In one or more embodiments, the electronic apparatus 100 may receive information about the user's exercise performance ability from the external device 10.

The electronic apparatus 100 may update the exercise routine based on the exercise performance ability. Specifically, the electronic apparatus 100 may update information about the exercise routine based on whether the user has achieved the goals of the exercise routine and the user's level of fatigue. For example, when the user fails to complete the number of exercises or the user's fatigue level has increased, the electronic apparatus 100 may update the exercise routine to lower the intensity of the next exercise, reduce the number of exercises, or increase the rest period during the exercise routine.

In one or more embodiments, the electronic apparatus 100 may transmit information about the updated exercise routine to the external device 10. The external device 10 may provide the information about the updated exercise routine to the user.

When the exercise is finished, the electronic apparatus 100 may provide the user with statistical data on the performance results of the exercise routine. In addition, the electronic apparatus 100 may create the next exercise routine based on the performance results of the exercise routine.

As described above, by generating and updating the exercise routine based on the user data, the user's exercise performance ability can be improved.

FIG. 4 is a block diagram illustrating configuration of an electronic apparatus according to an embodiment. As shown in FIG. 4, the electronic apparatus 100 includes the at least one processor 110, the memory 120, the display 140, and the sensor 170. Meanwhile, the example shown in FIG. 4 is only one embodiment, and other configurations may be added or some configurations may be deleted. Further, portions of the description of FIG. 4 that are redundant with FIG. 1 will be omitted.

The memory 120 may store at least one instruction regarding the electronic apparatus 100. In addition, the memory 120 may store an operating system (O/S) for driving the electronic apparatus 100. In addition, the memory 120 may store various software programs or applications for operating the electronic apparatus 100 according to various embodiments. Specifically, the memory 120 may store various software modules for operating the electronic apparatus 100 according to various embodiments, and the at least one processor 110 may control the operation of the electronic apparatus 100 by executing various software modules stored in the memory 120. In order words, the memory 120 is accessed by the at least one processor 110, and data reading/writing/modifying/deleting/updating may be performed by the at least one processor 110.

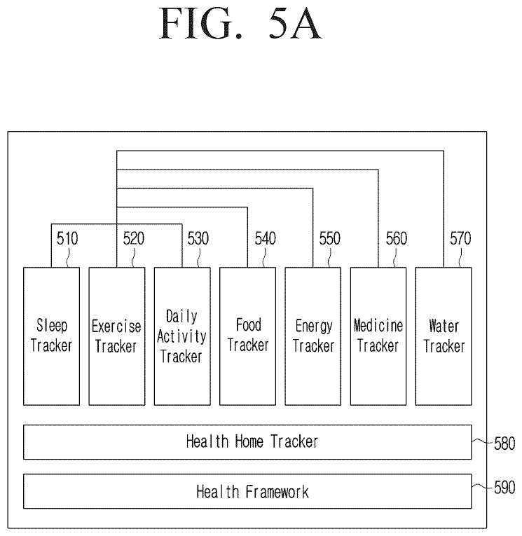

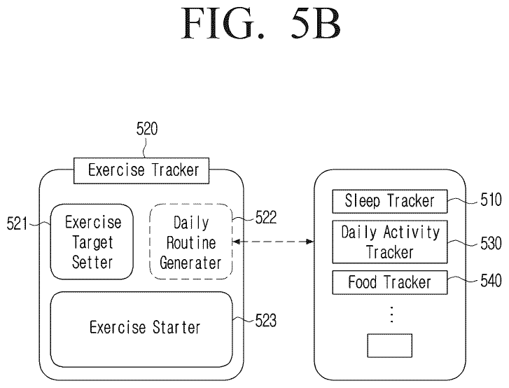

In one or more embodiments, the memory 120 may include a sleep tracker 510, an exercise tracker 520, a daily activity tracker 530, a food tracker 540, an energy tracker 550, a medicine tracker 560, a water tracker 570, a health home tracker 580, and a health framework 590, as shown in FIG. 5A. Here, the sleep tracker 510 is an application (or program) for managing the user's sleep, the exercise tracker 520 is an application for managing the user's exercise, the daily activity tracker 530 is an application for recording and managing daily activities, the food tracker 540 is an application for storing or managing information about the user's meals, and the energy tracker 550 is an application for managing the user's energy consumption, the medicine tracker 560 is an application for managing the user's medication information, the water tracker 570 is an application for managing the user's intake and discharge of water, the health home tracker 580 performs a function of managing applications 510 to 570 related to the user's health, and the health framework 590 is a developer tool and interface related to the health-related applications 510 to 570.

In particular, the exercise tracker 520 may include an exercise target setter 521, a daily routine generator 522, and an exercise starter 523, as shown in FIG. 5B. The exercise target setter 521 may perform a function of allowing the user to set an exercise goal. In this case, the exercise goal may include exercise distance, exercise time, calorie consumption, and exercise repetition number. For example, the exercise target setter 521 may set goals such as “run 3 km”, “ride a bike for 30 minutes”, “burn 200 calories”, or “repeat push-ups 10 times”. The daily routine generator 522 may perform a function of recommending an exercise routine to the user. In this case, the daily routine generator 522 may obtain the user data from health-related applications such as the sleep tracker 510, the daily activity tracker 530, and the food tracker 540, and may create an exercise routine for the user based on the user data. Further, the daily routine generator 522 may monitor the user's exercise performance ability or the user's progress, and may adjust (or update) the exercise routine as needed. The exercise starter 523 may perform a function of starting and tracking individual exercise according to the exercise goal set by the user. The exercise starter 523 may perform a function of collecting data suitable for the exercise goal according to the exercise goal selected by the user.

In addition, the memory 120 may store data (e.g., weight data) for calculating the condition score.

Further, the memory 120 may store a trained neural network model. In this case, the neural network model may be an artificial intelligence model trained to create an exercise routine for the user by inputting the user data.

The display 140 is a display device, which may display a graphical user interface (GUI) of application, function or service. The display 140 may have a touch panel superimposed or integrated at least in part or in its entirety, which may include electrode sensing elements for biosensing through touch, pressure sensing or transparent electrodes. In addition, the display 140 may include devices such as liquid crystal displays (LCDs), organic light emitting displays (OLEDs), and micro LEDs.

Meanwhile, the display 140 may display query information (or query items) for obtaining user data. Further, the display 140 may display various user interfaces (UIs) for creating an exercise routine corresponding to the user. Further, the display 140 may display information about the exercise process while performing an exercise according to the exercise routine and information about the updated exercise routine.

The sensor 170 may obtain data about the environment surrounding the electronic apparatus 100 or the user using the electronic apparatus 100. The sensor 170 may include an illuminance sensor, an inertial sensor, a magnetic sensor, a barometric sensor, a biometric sensor, a temperature sensor, and an electrode sensor.

The illuminance sensor is a sensor that obtains a sensing value for the brightness of external light to control the luminance of the display 140. The inertial sensor is a sensor that detects inertia, such as an acceleration sensor or a gyroscope. The inertial sensor may be equipped with only an acceleration sensor (3-axis), or may be equipped with a 6-axis sensor including an acceleration sensor and a gyroscope. The inertial sensor may obtain a sensing value for motion, gesture, impact, posture, and activity (sedentary, moving, sports) of the electronic apparatus 100. The magnetic sensor is a sensor detects external magnetic force, and may obtain a sensing value for measuring orientation by detecting the earth's magnetic field. The barometric sensor is a sensor that detects air pressure, and altitude can be estimated using the barometric sensor. The biometric sensor is a sensor that irradiates light to a living body and receives the light that is absorbed, scattered, and reflected. The emitter of the biometric sensor emits light of various bands, and may consist of elements such as LEDs, lasers, and VCSELs (vertical cavity surface emitting lasers). The band of the emitter may consist of various wavelengths such as green, red, infrared (IR), blue, yellow, and ultraviolet (UV). The receiver of the biometric sensor may receive the light reflected or transmitted after being irradiated from the emitter and store the value converted through an analog to digital converter (ADC) in the memory 120 or in a sensor buffer. The receiver of the biometric sensor may consist of a photodiode (PD) and a complementary metal-oxide-semiconductor (CMOS) (camera). The receiver of the biometric sensor may have a filter to receive light of a specific band or filter out light outside of a specific band. The controller of the biometric sensor may be an IC or an analog front-end (AFE), and may control the emitter and the receiver, process the received data, and transmit it to the at least one processor 110 or store it in the memory 120. In addition, the biometric sensor may emit sound waves instead of light to a living body and detect a target. Alternatively, the biometric sensor may emit light and receive light that is absorbed, scattered, or reflected, emit sound waves and receive sound waves that are reflected, or sense images in various combinations. The biometric sensor may include a photoplethysmogram (PPG) sensor that detects pulse waves with light, and may measure heart rate (HR), heart rate variability (HRV), blood oxygen saturation (SpO2), and blood pressure. In addition, the biometric sensor may include a biomarker sensor that detects a specific substance or component in the body. The biomarker is an indicator that may detect changes in the body, such as cells, blood vessels, proteins, deoxyribo nucleic acid (DNA), ribo nucleic acid (RNA), and metabolites in the body, and may detect blood sugar, alcohol, advanced glycation end-product (AGE), and antioxidants. The temperature sensor is a sensor that measures the temperature of a living body or a part. Depending on the method, the temperature sensor may be a contact type or a non-contact type. The temperature value measured by the temperature sensor may be stored in the memory 120, or transmitted to the processor 110 and used to estimate a skin temperature, recognize a situation or estimate body temperature.

The electrode sensor is a sensor having an electrode that senses the characteristics of a living body through contact with the living body. The electrode is an interface for measuring electrical characteristics (voltage, current, impedance) through the living body, and may detect various physical characteristics by forming an equivalent circuit through the body. In the case of a smart watch, the living body condition can be measured by detecting electrical signals generated from body activities through an electrocardiogram (ECG), an electromyogram (EMG), and an electroencephalogram (EEG). In the case of a smart watch, the electrical signals generated from the heart can be measured using the INP (wrist) and INM electrodes (finger of the opposite hand) for ECG measurement. In addition, the RLD electrode can be used as a ground to match the potential standard of the living body signal, thereby increasing the accuracy of the living body signal measurement. The four electrodes of the smart watch may be used to measure body composition through body impedance analysis (BIA) measurement. The body composition may include body fat and body water. The two electrodes of the smart watch may be used to measure the electrodermal activity (EDA) of the skin. The EDA may include all measurements related to skin electrical response such as skin conductance, galvanic skin response (GSR), electrodermal response (EDR), and psychogalvanic reflex (PRG). As such, electrode sensors may measure various biomarkers based on sensor circuits that are connected to the body through electrodes as an interface, and generate bio-related data.

The at least one processor 110 may control the electronic apparatus 100 in accordance with at least one instruction stored in the memory 120.

In particular, the at least one processor 110 may include one or more processors. Specifically, the one or more processors may include one or more of a central processing unit (CPU), a graphics processing unit (GPU), an accelerated processing unit (APU), a many integrated core (MIC), a digital signal processor (DSP), a neural processing unit (NPU), a hardware accelerator, or a machine learning accelerator. The one or more processors may control one or any combination of other components of the electronic apparatus, and may perform operations related to communication or data processing. The one or more processors may execute one or more programs or instructions stored in the memory. For example, the one or more processors may perform a method according to an embodiment of the present disclosure by executing one or more instructions stored in the memory. For example, the processor 110 may correspond to a plurality of processors that collectively perform a plurality of operations by dividing them among the processors.

When a method according to an embodiment includes a plurality of operations, the plurality of operations may be performed by one processor or by a plurality of processors. In other words, when a first operation, a second operation, and a third operation are performed by the method according to an embodiment, all of the first operation, the second operation, and the third operation may be performed by the first processor, or the first operation and the second operation may be performed by the first processor (e.g., a general-purpose processor) and the third operation may be performed by the second processor (e.g., an artificial intelligence-dedicated processor). For example, according to an embodiment, the operation of creating an exercise routine using a neural network model may be performed by a processor that performs parallel operations, such as a GPU or an NPU, and the operation of measuring exercise performance ability or calculating a condition score may be performed by a general-purpose processor such as a CPU.

The one or more processors may be implemented as a single core processor including a single core, or as one or more multicore processors including a plurality of cores (e.g., homogeneous multicore or heterogeneous multicore). When the one or more processors 110 are implemented as a multicore processor, each of the plurality of cores included in the multicore processor may include internal memory of the processor, such as cache memory and an on-chip memory, and a common cache shared by the plurality of cores may be included in the multicore processor. Each of the plurality of cores (or some of the plurality of cores) included in the multi-core processor may independently read and perform program instructions to implement the method according to an embodiment, or all (or some) of the plurality of cores may be coupled to read and perform program instructions to implement the method according to an embodiment.

When a method according to an embodiment includes a plurality of operations, the plurality of operations may be performed by one core of a plurality of cores included in a multi-core processor, or may be performed by a plurality of cores. For example, when a first operation, a second operation, and a third operation are performed by a method according to an embodiment, all of the first operation, the second operation, and the third operation may be performed by the first core included in the multi-core processor, or the first operation and the second operation may be performed by the first core included in the multi-core processor and the third operation may be performed by the second core included in the multi-core processor.

In the embodiments of the present disclosure, the at least one processor 110 may mean a system-on-chip (SoC) in which at least one processor and other electronic components are integrated, a single-core processor, a multi-core processor, or a core included in a single-core processor or multi-core processor, and here, the core may be implemented as CPU, GPU, APU, MIC, DSP, NPU, hardware accelerator, or machine learning accelerator, etc., but the core is not limited to the embodiments of the present disclosure.

In particular, the at least one processor 110 may, by the at least one instruction stored in the memory 120, control the electronic apparatus 100 to obtain data about the user, obtain information about an exercise routine corresponding to the user based on the obtained data, obtain information about the user's exercise performance ability while the user is performing the exercise using the exercise routine, and update the exercise routine based on the exercise performance ability.

In one or more embodiments, the at least one processor 110 may provide query information for obtaining information about at least one of a current condition or an exercise ability of a user, and may obtain data about the user in response to a user input entered based on the query information. Here, the query information may include at least one of information about the user's diet, information about the user's sleep, information about the user's alcohol consumption, information about the user's medication, information about the user's activity amount, information about the user's body fat, information about the user's targeted exercise area, and information about the user's satisfaction with an existing exercise routine.

In one or more embodiments, the at least one processor 110 may, based on the obtained data, obtain information about whether the user is able to perform exercise and information about exercise intensity, identify an exercise goal information about whether the user is able to perform exercise and information about exercise intensity, analyze feedback information about the existing exercise routine, and obtain information about an exercise routine corresponding to the user based on the exercise goal and the feedback information.

In one or more embodiments, when the user drinks alcohol within a preset drinking period, takes a preset drug within a preset drug period, eats less than a threshold amount of food, sleeps less than a threshold sleeping time, or has a vitality score less than a preset vitality value, the at least one processor 110 may obtain information about an exercise routine of first intensity, when the user does not drink alcohol within the preset drinking period, does not take the preset drug within the preset drug period, eats more than the threshold amount of food, sleeps more than the threshold sleeping time, or has the vitality score more than the preset vitality value, the at least one processor 110 may obtain information about an increase in body fat, when the increase in body fat is less than a preset body fat value, the at least one processor 110 may obtain an exercise routine of second intensity, and when the increase in body fat is grater than the preset body fat value, the at least one processor 110 may obtain an exercise routine of third intensity. Here, the first intensity may be lower than the second intensity, and the second intensity may be lower than the third intensity.

In one or more embodiments, the at least one processor 110 may calculate a condition score based on information about the user's meal amount, the user's last meal time, the user's sleep amount, whether the user drinks alcohol, the user's activity amount, the user's change in body fat, and a degree of performance of the existing exercise routine, and obtain information about an exercise routine corresponding to the user by adjusting the exercise goal of the existing exercise routine based on the existing exercise routine and the condition score.

In one or more embodiments, the at least one processor 110 may use the exercise routine to obtain information about the user's condition and progress in the exercise routine while the user is performing the exercise, and may update detailed settings of the next exercise included in the exercise routine based on the information about the user's condition and progress in the exercise routine.

In one or more embodiments, the exercise routine includes a plurality of exercises and a rest period between the plurality of exercises, and the at least one processor 110 may provide information about detailed settings of the next exercise updated during the rest period. Here, the detailed settings of the exercise may include at least one of the number of progressions of the exercise, a weight of a tool required for the exercise, or the number of sets of the exercise.

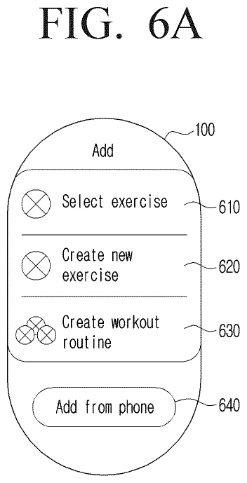

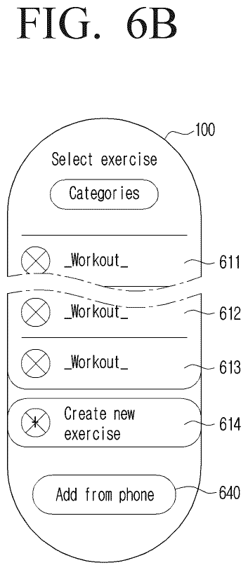

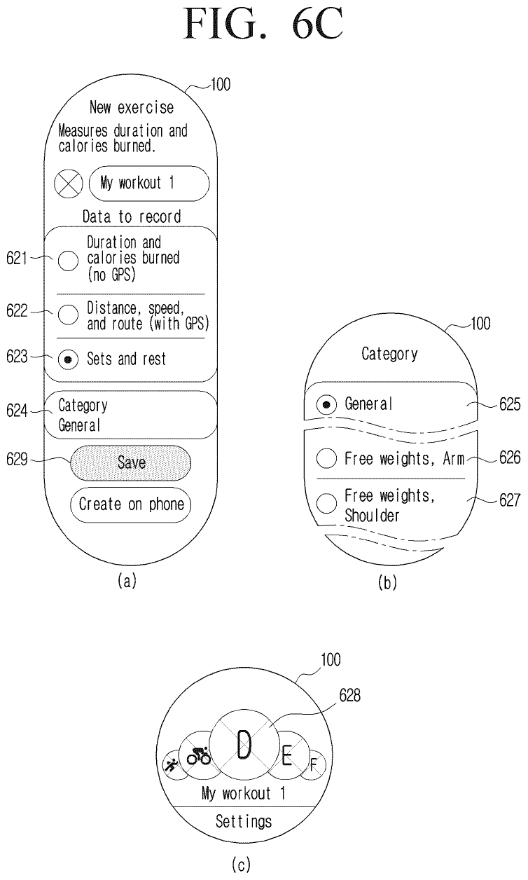

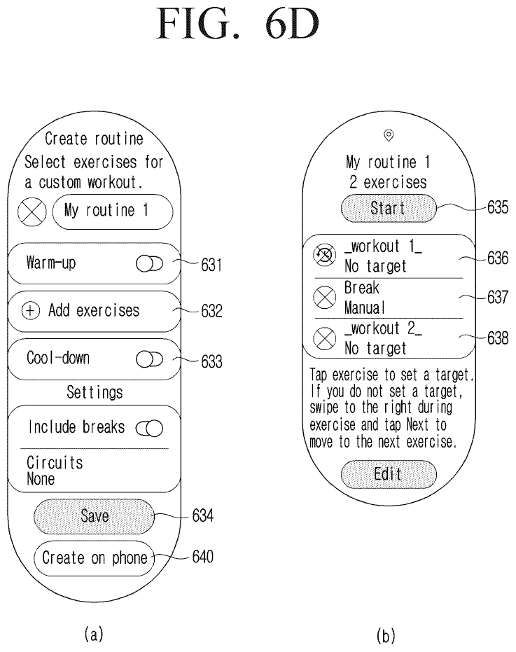

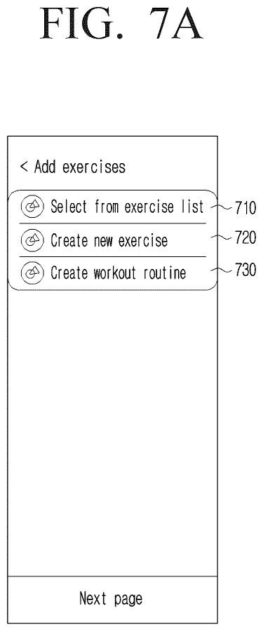

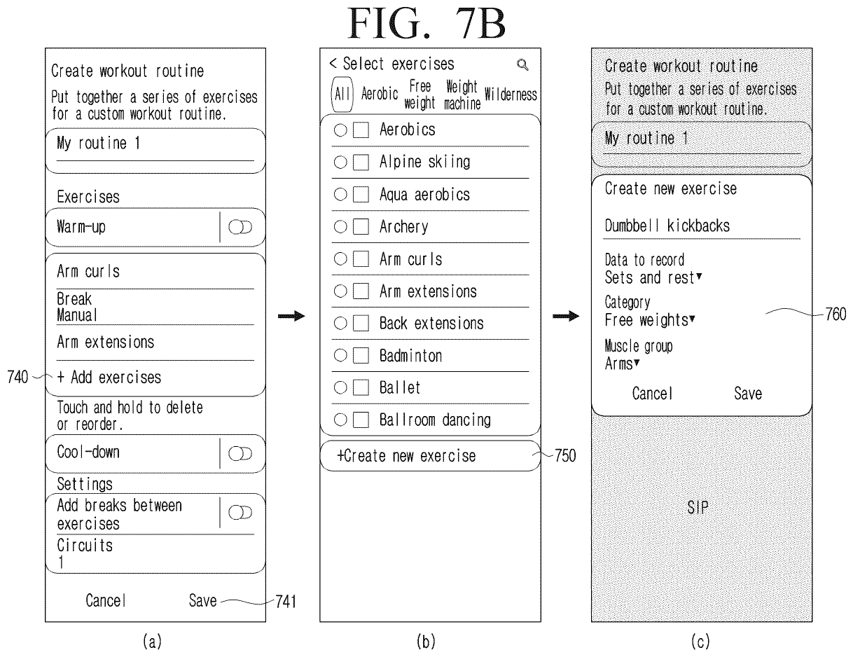

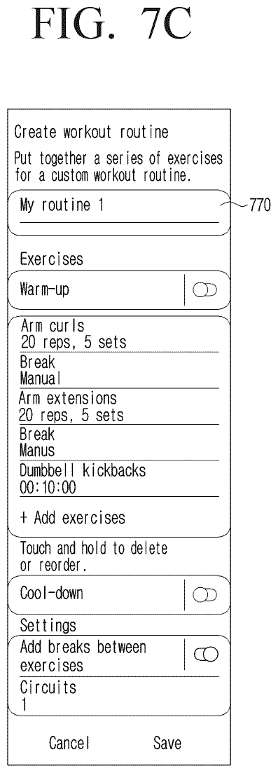

Meanwhile, the electronic apparatus 100 may provide a UI for creating an exercise routine in response to a user input for creating an exercise routine. This will be described with reference to FIGS. 6A to 7C.

FIGS. 6A to 6D are views provided to explain a method of generating an exercise or an exercise routine using an electronic apparatus according to an embodiment. Meanwhile, in this case, only a portion of the UI illustrated in FIGS. 6A to 6D may be displayed via display 140, and the UI illustrated in FIG. 6A may be scrolled in response to a user input for scrolling the UI.

First, when a user command to select an application for creating an exercise routine is entered, the electronic apparatus 100 may provide a UI as shown in FIG. 6A. The UI shown in FIG. 6A may include a plurality of UI elements 610 to 640 for generating an exercise or an exercise routine. Here, the first UI element 610 is a UI element for selecting an exercise, the second UI element 620 is a UI element for generating a new exercise, the third UI element 630 is a UI element for generating a new exercise routine, and the fourth UI element 640 is a UI element for obtaining an exercise or an exercise routine from the external device 10. Here, the UI elements may be replaced by terms such as items, icons, indicators, and the like.

When the first UI element 610 is selected, the electronic apparatus 100 may be provided with a UI as shown in FIG. 6B. The screen shown in FIG. 6B may include a plurality of UI elements 611 to 613 corresponding to predefined exercises, a UI element 614 for generating a new exercise, and a UI element 640 for obtaining an exercise from the external device 10.

When the second UI element 620 is selected, the electronic apparatus 100 may provide a UI including UI elements 621 to 623 for generating a new exercise and a UI element 624 for setting a category, as shown in (a) of FIG. 6C. Here, when the UI element 624 for setting a category is selected, the electronic apparatus 100 may provide a UI including a plurality of UI elements 625 to 627 corresponding to a plurality of categories, as shown in (b) of FIG. 6C. Alternatively, when a Save UI element 629 is selected after one of the plurality of exercises is selected, the electronic apparatus 100 may provide a UI including a UI element 628 corresponding to the selected exercise, as shown in (c) of FIG. 6C.

When the third UI element 630 is selected, the electronic apparatus 100 may provide a UI for creating an exercise routine, as shown in (a) of FIG. 6D. The UI for creating an exercise routine may include a UI element 631 for generating a warm-up exercise, a UI element 632 for adding an exercise to the exercise routine, and a UI element 633 for generating a cool-down exercise. When a Save UI element 634 is selected after the exercise routine is created using the plurality of UI elements 631 to 633, the electronic apparatus 100 may be provided with a UI including information about the exercise routine set by the user, as shown in (b) of FIG. 6D. The UI including information about the exercise routine may include UI elements 636 to 638 indicating a plurality of exercises and rest periods included in the exercise routine, and a UI element 635 for starting the exercise routine.

As described above, the electronic apparatus 100 may set an exercise and an exercise routine in response to a user input.

According to one or more embodiments, the electronic apparatus 100 may receive information about the exercise or exercise routine obtained through a UI provided via the external device 10. FIGS. 7A to 7C are views provided to explain a method of generating an exercise or an exercise routine using an external device according to an embodiment.

First, when a user command to select an application for creating an exercise routine is entered, the external device 10 may provide a UI as shown in FIG. 7A. The UI shown in FIG. 7A may include a plurality of UI elements 710 to 730 for generating an exercise or an exercise routine. Here, the first UI element 710 is a UI element for selecting an exercise, the second UI element 720 is a UI element for generating a new exercise, and the third UI element 730 is a UI element for generating a new exercise routine.

Since the first UI element 710 and the second UI element 720 perform the same functions as described in FIG. 6A, redundant description will be omitted.

When the first UI element 730 is selected, the external device 10 may be provided with a UI for creating an exercise routine, as shown in (a) of FIG. 7B. The screen shown in FIG. 7B may include a plurality of UI elements for creating an exercise routine. For example, the screen may include a UI element for generating a warm-up exercise, a UI element for generating a plurality of exercises and rest periods, and a UI element for generating a cool-down exercise.

In particular, when a UI 740 for generating an exercise to be included in an exercise routine is selected, the external device 10 may provide a UI including a plurality of UI elements representing predefined exercises, as shown in (b) of FIG. 7B. The UI may further include a UI element 750 for generating a new exercise. When the UI element 750 is selected, the external device 10 may provide a screen 760 for generating a new exercise, as shown in (c) of FIG. 7B.

Further, when the exercises and rest periods to be included in the exercise routine are set via the UI of (a) in FIG. 7B, the external device 10 may provide a UI 770 indicating the exercises and rest periods included in the exercise routine, as shown in FIG. 7C. In this case, when a Save UI element 741 is selected, the external device 10 may store information about the exercise routine, and may transmit the information about the exercise routine to the electronic apparatus 100 in response to a user input.

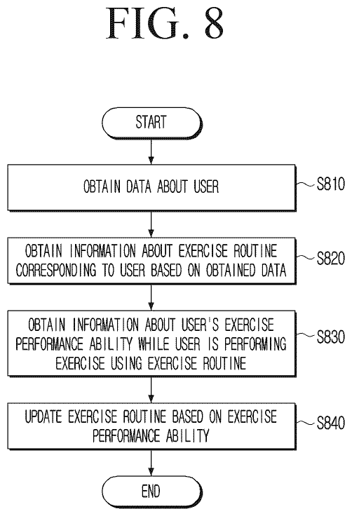

FIG. 8 is a flowchart provided to explain a method of providing and updating an exercise routine based on data about a user according to an embodiment.

In the following embodiments, each operation may be performed sequentially, but is not necessarily performed sequentially. For example, the order of each operation may be changed, and at least two operations may be performed in parallel.

According to an embodiment, it can be understood that S810 to S840 are performed in a processor (e.g., the processor 110 of FIG. 4) of an electronic apparatus (e.g., the electronic apparatus 100 of FIG. 3).

First, according to an embodiment, the electronic apparatus 100 obtains data about a user (S810). The data about the user is data for determining the user's current physical condition and exercise ability, and may include data about the user's profile, data about the user's physical information, data about the user's condition, data about the user's exercise experience, data about the user's injury history, data about the user's physical strength, and data about the user's preferred type of exercise.

In one or more embodiments, the electronic apparatus 100 may obtain the user's profile, such as age information and gender information, stored in the memory 120 of the electronic apparatus 100.

In one or more embodiments, the electronic apparatus 100 may obtain data about the user's physical information from the sensor 170 of the electronic apparatus 100. For example, the electronic apparatus 100 may obtain the physical information such as information about the user's pulse rate, information about blood pressure, information about electrocardiogram, information about electromyogram, and information about electroencephalogram through the sensor 170.

In one or more embodiments, the electronic apparatus 100 may obtain data about the user's exercise experience stored in the memory 120, data about the user's injury history, data about the user's physical strength, and data about the type of exercise the user prefers.

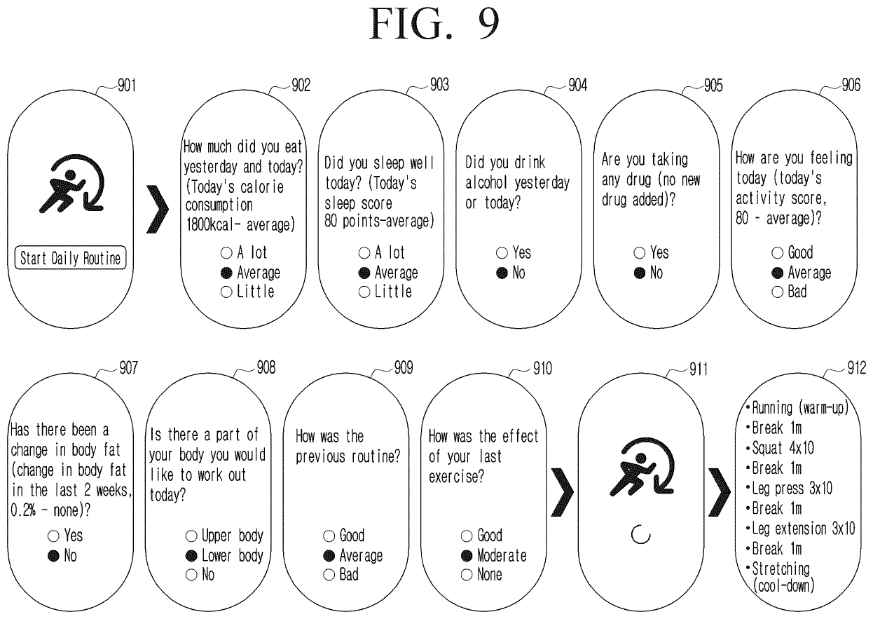

In one or more embodiments, the electronic apparatus 100 may use query information to obtain user data. This will be described with reference to FIG. 9. FIG. 9 depicts views illustrating query items for obtaining data about a user according to an embodiment.

Specifically, the electronic apparatus 100 may provide a first screen 901 for initiating query information to set an exercise routine. Once a Get Started UI element is selected on the first screen 901, the electronic apparatus 100 may provide a second screen 902 obtaining information about the user's meal amount. When the meal amount information is input through the second screen 902, the electronic apparatus 100 may provide a third screen 903 for obtaining information about the user's sleep amount. When the sleep amount information is input through the third screen 903, the electronic apparatus 100 may provide a fourth screen 904 for obtaining information about whether the user drinks alcohol. When the alcohol consumption information is input through the fourth screen 904, the electronic apparatus 100 may provide a fifth screen 905 for obtaining the user's medication information. When the medication information is input through the fifth screen 905, the electronic apparatus 100 may provide a sixth screen 906 for obtaining the user's physical condition information. When the condition information is input through the sixth screen 906, the electronic apparatus 100 may provide a seventh screen 907 for obtaining information about the user's body fat change. When the information about the body fat change is input through the seventh screen 907, the electronic apparatus 100 may provide an eighth screen 908 for obtaining information about the user's desired exercise area. When the information about the user's desired exercise area is input through the eighth screen 908, the electronic apparatus 100 may provide a ninth screen 909 for obtaining feedback information about the user's previous exercise routine. When the feedback information about the previous exercise routine is input through the ninth screen 909, the electronic apparatus 100 may provide a tenth screen 910 for obtaining exercise effectiveness information about the user's previous exercise routine. When the exercise effectiveness information is input through the tenth screen 910, the electronic apparatus 100 may provide an eleventh screen 911 that is displayed while creating an exercise routine based on the user data input through the setting information. After the eleventh screen 911 is displayed, the electronic apparatus 100 may provide a twelfth screen 912 that includes information about the exercise routine obtained based on the user data.

However, the operation of entering user data through the query information shown in FIG. 9 may be omitted depending on the user's choice.

Referring back to FIG. 8, the electronic apparatus 100 obtains information about an exercise routine corresponding to the user based on the obtained data (S820).

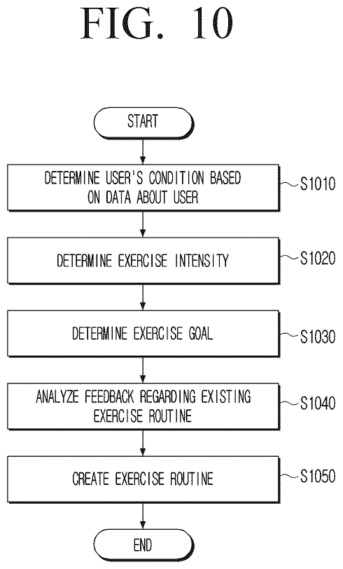

According to one or more embodiments, the electronic apparatus 100 may obtain an exercise routine corresponding to the user through a rule-based generation algorithm. This will be described with reference to FIGS. 10 to 12.

FIG. 10 is a flowchart provided to explain a method of creating an exercise routine based on data about a user according to an embodiment.

In the following embodiments, each operation may be performed sequentially, but is not necessarily performed sequentially. For example, the order of each operation may be changed, and at least two operations may be performed in parallel.

According to an embodiment, it can be understood that S1010 to S1050 are performed in a processor (e.g., the processor 110 in FIG. 4) of an electronic apparatus (e.g., the electronic apparatus 100 in FIG. 3).

First, according to an embodiment, the electronic apparatus 100 may determine the user's condition based on data about the user (S1010). Specifically, in step S810, the electronic apparatus 100 may obtain level information about whether the user's condition is good or bad based on the obtained user data, such as meals, sleep, vitality score, drinking, injury, and medication. For example, the electronic apparatus 100 may determine a first level or a third level based on the user's condition.

The electronic apparatus 100 may determine an exercise intensity (S1020). Specifically, the electronic apparatus 100 may determine the exercise intensity according to a level based on the user's condition. For example, when the user's condition is identified as the first level, the electronic apparatus 100 may determine the exercise intensity to be first intensity, when the user's condition is identified as the second level, the electronic apparatus 100 may determine the exercise intensity to be second intensity, and when the user's condition is identified as the third level, the electronic apparatus 100 may determine the exercise intensity to be third intensity.

The electronic apparatus 100 may determine an exercise goal (S1030). Here, the exercise goal may be an exercise area or exercise type desired by the user. For example, the electronic apparatus 100 may determine whether the exercise area desired by the user as an exercise goal is the lower body, the upper body, or the whole body. In addition, the electronic apparatus 100 may determine whether the exercise type desired by the user as an exercise goal is aerobic or anaerobic.

The electronic apparatus 100 may analyze feedback about the existing exercise routine (S1040). Specifically, the electronic apparatus 100 may analyze information about whether the existing exercise routine is preferred, the effectiveness of the existing exercise routine, etc.

The electronic apparatus 100 may create an exercise routine (S1050). Specifically, the electronic apparatus 100 may create the exercise routine based on the exercise intensity, exercise goal, and feedback about the existing exercise routine obtained through steps S1020 to S1040. For example, the electronic apparatus 100 may adjust the number of sets, number of repetitions, and duration of the exercise based on the exercise intensity. In addition, the electronic apparatus 100 may adjust the exercises included in the exercise routine based on the exercise goal. Further, the electronic apparatus 100 may determine whether to use exercises from the existing exercise routine based on feedback about the existing exercise routine. In other words, when the feedback about the existing exercise routine is good and the existing exercise is identified as effective, the electronic apparatus 100 may create an exercise routine based on the information about the existing exercise routine. When the feedback about the existing exercise routine is not good or the existing exercise is identified as ineffective, the electronic apparatus 100 may create an exercise routine by changing some of the exercises included in the existing exercise routine.

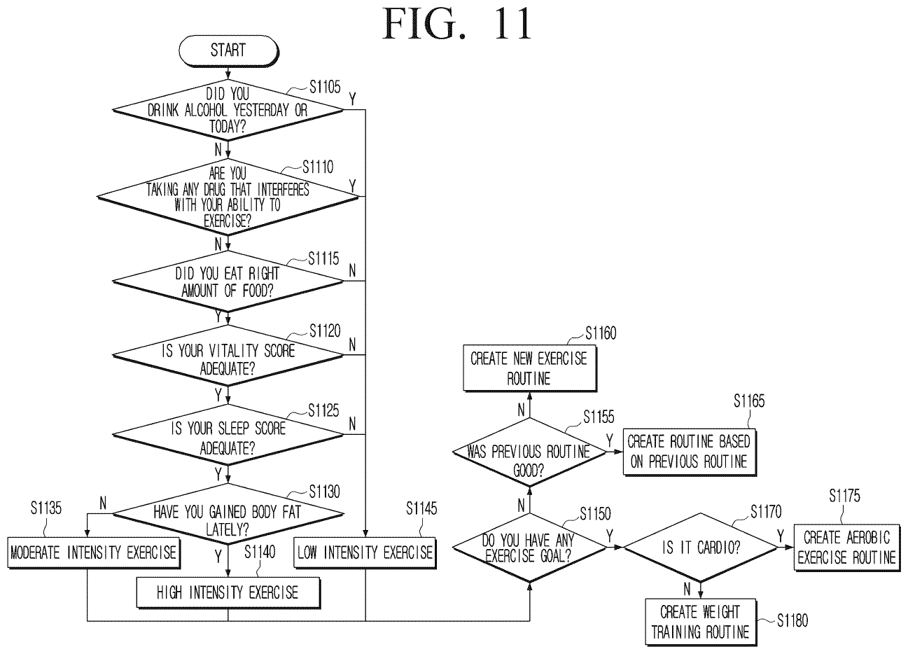

FIG. 11 is a flowchart provided to specifically explain a method of creating an exercise routine based on data about a user according to an embodiment.

In the following embodiments, each operation may be performed sequentially, but is not necessarily performed sequentially. For example, the order of each operation may be changed, and at least two operations may be performed in parallel.

According to an embodiment, it can be understood that S1105 to S1180 are performed in a processor (e.g., the processor 110 of FIG. 4) of an electronic apparatus (e.g., the electronic apparatus 100 of FIG. 3).

First, according to an embodiment, the electronic apparatus 100 may identify whether the user drank alcohol the previous day or today (S1105). In other words, the electronic apparatus 100 may identify whether the user drank alcohol within a preset drinking time period (e.g., the previous day or today) based on drinking data among the user data.

When it is identified that the user drank alcohol the previous day or today (S1105-Y), the electronic apparatus 100 may identify the exercise intensity of the exercise routine as a low-intensity exercise (S1145).

When it is identified that the user did not drink alcohol the previous day or today (S1105-Y), the electronic apparatus 100 may identify whether the user is taking a drug that could interfere with exercise (S1110). In other words, the electronic apparatus 100 may identify whether the user has taken a drug within a preset drug period (e.g., the previous day or today) based on the medication data among the user data.

When it is identified that the user is taking a drug that interferes with exercise (S1110-Y), the electronic apparatus 100 may identify the exercise intensity of the exercise routine as a low intensity exercise (S1145).

When it is identified that the user is not taking any drug that interferes with exercise (S1110—N), the electronic apparatus 100 may identify whether the user has eaten an adequate amount of food (S1115). In other words, the electronic apparatus 100 may identify whether the user's meal amount is less than a threshold amount based on the meal amount data among the user data. In addition, based on whether the meal amount is less than the threshold amount, the electronic apparatus 100 may identify whether the user has eaten a moderate amount of the meal.

When it is identified that the user has not eaten an adequate amount of food (S1115—N), the electronic apparatus 100 may identify the exercise intensity of the exercise routine as a low intensity exercise (S1145).

When it is identified that the user has eaten an adequate amount of food (S1115-Y), the electronic apparatus 100 may identify whether the user has an adequate vitality score (S1120). Here, the vitality score may be a score that specifically indicates the user's physical strength calculated by utilizing the sleep data, the user's daily activity amount, and weekly exercise. The electronic apparatus 100 may identify whether the vitality score is adequate based on whether the vitality score is more than a preset vitality value.

When it is identified that the vitality score is not adequate (S1120—N), the electronic apparatus 100 may identify the exercise intensity of the exercise routine as a low intensity exercise (S1145).

When it is identified that the vitality score is adequate (S1120—Y), the electronic apparatus 100 may identify whether the sleep score is adequate (S1125). Here, the sleep score may be a score calculated by sleep quantity, sleep quality, etc. The electronic apparatus 100 may identify whether the sleep score is adequate based on whether the sleep score (or sleep quantity) is less than a threshold sleeping value.

When it is identified that the sleep score is not adequate (S1125—N), the electronic apparatus 100 may identify the exercise intensity of the exercise routine as a low intensity exercise (S1145).

When it is identified that the sleep score is adequate (S1125—Y), the electronic apparatus 100 may identify whether the user's body fat has increased (S1130). In other words, the electronic apparatus 100 may obtain information about the amount of increase in body fat using body fat data among the user data. In particular, the electronic apparatus 100 may identify whether the user's body fat has increased by identifying whether the amount of increase in body fat is more than a preset body fat value.

When it is identified that the user's body fat has not increased (i.e., when the user's body fat has decreased or increased less than a preset value) (S1130—N), the electronic apparatus 100 may identify the exercise intensity of the exercise routine as a moderate intensity exercise (S1135).

When it is identified that the user's body fat has increased (i.e., when the user's body fat has increased more than a preset body fat value) (S1130—Y), the electronic apparatus 100 may identify the exercise intensity of the exercise routine as a high intensity exercise (S1140).

After identifying the intensity of the exercise, such as in steps S1105 to S1145, the electronic apparatus 100 may identify whether the user has a targeted exercise (S1150). Here, the exercise that the user is targeting may be one of aerobic exercise or anaerobic exercise (e.g., strength training).

When it is identified that the user does not have a targeted exercise (S1150—N), the electronic apparatus 100 may identify whether the configuration of the previous routine was good (S1155).

When it is identified that the user likes the configuration of the previous exercise routine (S1155—Y), the electronic apparatus 100 may create an exercise routine based on the previous routine (S1165).

When it is identified that the user does not like the configuration of the previous exercise routine (S1155—N), the electronic apparatus 100 may create a new exercise routine (S1160).

When it is identified that the user has a targeted exercise (S1150—Y), the electronic apparatus 100 may identify whether the exercise the user is targeting is aerobic (S1170).

When the user's targeted exercise is aerobic (S1170—Y), the electronic apparatus 100 may create an aerobic-based exercise routine (S1175).

When the exercise the user is targeting is not aerobic (S1170—Y), the electronic apparatus 100 may create a strength training routine (S1180).

In one or more embodiments, the electronic apparatus 100 may calculate a condition score based on information about the user's meal amount, information about the user's last meal time, information about how much the user's sleep amount, information about whether the user has drank alcohol, information about the user's activity amount, information about the user's body fat change, and the user's degree of performance of the existing exercise routine.

Specifically, the electronic apparatus may calculate a condition score based on the following criteria.
1) Meal amount (10%)
   0 points if it is most different from the usual meal amount
   100 points if it is the closest to the usual meal amount
2) Last meal time (10%)
   0 points if it is not different from the last meal time
   100 points if it is most different from the last meal time
3) Sleep score (20%)
   0 for the worst quality of sleep
   100 for the best quality of sleep
4) Alcohol consumption (10%)
   0 points for drinking alcohol
   100 points for not drinking alcohol
5) Medication status (10%)
   0 points if on medication
   100 points if not on medication
6) Activity score (30%)
   0 if it is the lowest compared to normal
   100 points if it is the highest compared to normal
7) Body fat change (5%)
   0 points if body fat increases the most
   100 points if body fat decreases the most
8) Past performance of similar routine (5%)
   0 if no past similar routine performance experience
   100 points if a lot of past similar routine performance experience

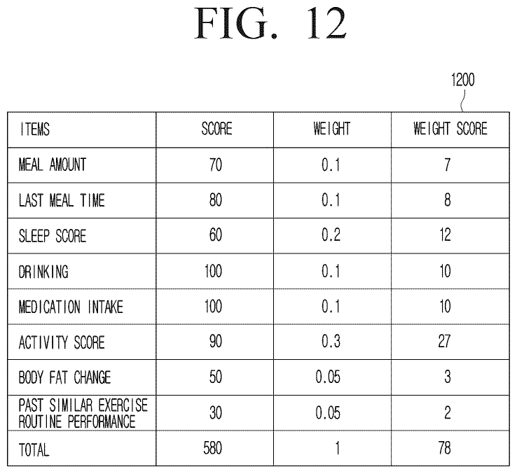

Subsequently, the electronic apparatus 100 may calculate a final condition score by multiplying the score of each item by a weight, as shown in table 1200 in FIG. 12. For example, the electronic apparatus 100 may obtain a weighted score (7 points) by multiplying the meal amount score (70 points) by a weight (0.1), obtain a weighted score (8 points) by multiplying the last meal time score (80 points) by a weight (0.1), obtain a weighted score (12 points) by multiplying the sleep score (60 points) by a weight (0.2), obtain a weighted score (10 points) by multiplying the drinking score (100 points) by a weight (0.1), obtain a weighted score (10 points) by multiplying the medication score (100 points) by a weight (0.1), obtain a weighted score (27 points) by multiplying the activity score (90 points) by a weight (0.3), obtain a weighted score (3 points) by multiplying the body fat change score (50 points) by a weight (0.05), and obtain a weighted score (2 points) by multiplying the past similar exercise routine performance score (30 points) by a weight (0.05). In addition, the electronic apparatus 100 may obtain a condition score (78 points) by adding the obtained weighted scores.

Further, the electronic apparatus 100 may obtain information about an exercise routine corresponding to the user by adjusting an exercise goal (or exercise intensity) of the previous exercise routine based on the previous exercise routine and the condition score. In other words, when it is identified that the condition score is more than a preset value, the electronic apparatus 100 may obtain information about an exercise routine by using an exercise goal of the previous exercise routine, and when it is identified that the condition score is less than the preset value, the electronic apparatus 100 may obtain information about an exercise routine by adjusting the exercise goal of the previous exercise routine.

Meanwhile, in the above embodiment, it is described that an exercise routine is created (or obtained) through a rule-based algorithm, this is only one embodiment, and the electronic apparatus 100 may obtain an exercise routine corresponding to the user through a generative AI model. Here, the generative AI model may generally refer to an artificial intelligence neural network that relies on a user input for generating new forms of data. The generative AI models may include a model that generates images and/or a model that generates languages. Examples of the image generating model include generative adversarial networks (GANs), variational auto encoders (VAEs), and diffusion-based generative models that use VAEs and transformer structures. The language generating model is a model that is trained to produce the most statistically appropriate output based on an input value, such as CHAT-GPT 3 and CHAT-GPT 4. There are also large multimodal models (LMMs) that can recognize various forms of data input, such as text, images, and speech, and generate new data corresponding thereto. In particular, according to an embodiment, the electronic apparatus 100 may input user data into a generative AI model to obtain information about an exercise routine corresponding to the user.

Referring back to FIG. 8, the electronic apparatus 100 may use an exercise routine to obtain information about the user's exercise performance ability while the user is performing the exercise (S830). Here, the exercise performance ability may include at least one of whether the exercises included in the exercise routine have been performed, information about the user's level of fatigue, and information about changes in the user's body.

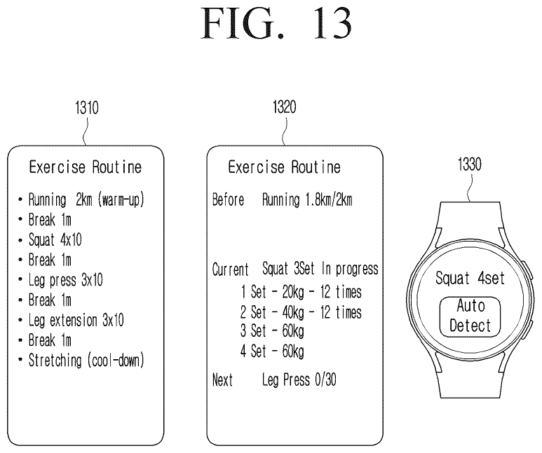

Specifically, the electronic apparatus 100 (or the external device 10) may provide information about an exercise routine, as shown in the first screen 1310 of FIG. 13. Here, the information about the exercise routine may include information about the types of exercises included in the exercise routine, the number of exercises, and rest periods. The electronic apparatus 100 (or the external device 10) may aggregate the user's performance history of the exercise routine while the user is performing the exercise using the exercise routine, and provide information about the performance history of the exercise routine, as shown in the second screen 1320 in FIG. 13. Further, the electronic apparatus 100 may provide information about a detect of whether the user has performed exercises while the user is performing exercises using the exercise routine, as shown in the third screen 1330 of FIG. 13.

The electronic apparatus 100 may update the exercise routine based on the exercise performance ability (S840). Specifically, the electronic apparatus 100 may update the exercise routine in real time based on whether the user has performed the exercise, information about the user's level of fatigue, and information about changes in the user's body.

In one or more embodiments, when the user fails to complete the number of repetitions or time of the first exercise included in the exercise routine, the electronic apparatus 100 may update the exercise routine to lower the exercise time or exercise repetition number of the second exercise following the first exercise included in the exercise routine, or update the exercise routine to increase the rest period between the first exercise and the second exercise included in the exercise routine.

In one or more embodiments, when the user's level of increases or a rapid change occurs in the user's body (e.g., a change in which the heart rate increases rapidly) while the user is performing the first exercise included in the exercise routine, the electronic apparatus 100 may update the exercise routine to lower the exercise time or exercise repetition number of the second exercise following the first exercise included in the exercise routine, or update the exercise routine to increase the rest period between the first exercise and the second exercise included in the exercise routine. Alternatively, the electronic apparatus 100 may provide a guide to instruct the user to rest.

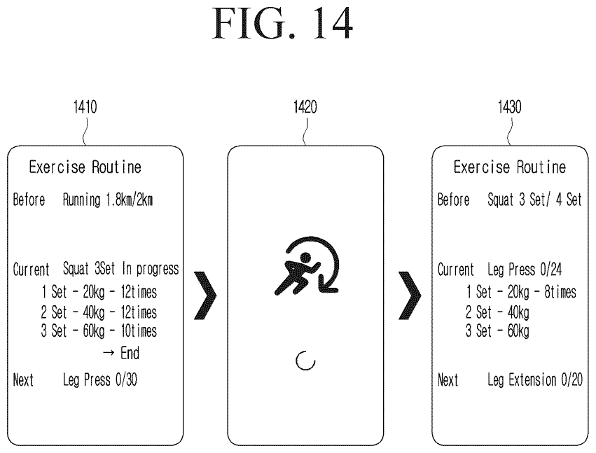

Here, the electronic apparatus 100 (or the external device 10) may provide information about an updated exercise routine. Specifically, as shown in the first screen 1410 of FIG. 14, the electronic apparatus 100 (or the external device 10) may provide a performance history of the exercise routine. Here, the electronic apparatus 100 (or the external device 10) may determine whether the exercise routine should be updated based on the exercise performance ability after the first exercise, and may provide a screen determining whether the exercise routine should be updated, as shown in the second screen 1420 of FIG. 14. When the exercise routine is updated, the electronic apparatus 100 (or the external device 10) may provide information about the updated exercise routine, as shown in the third screen 1430 of FIG. 14. In this case, the electronic apparatus 100 (or the external device 10) may provide information about the updated exercise routine. Here, the information about the updated exercise routine may be provided distinctively from the information about the existing exercise routine. For example, the information about the updated exercise routine may be displayed or highlighted in a different color from the information about the existing exercise routine.

According to the embodiment described above, the electronic apparatus 100 may create and provide an exercise routine that is appropriate for the user's condition and exercise preferences, and may update the exercise routine in real time based on the user's exercise performance ability, thereby increasing the effectiveness of the user's exercise and health management using the electronic apparatus 100.

Meanwhile, according to an embodiment, when the electronic apparatus 100 is implemented as a device without a display, such as a smart ring, the query information and information about the exercise routine can be provided via the external device 10.

In addition, according to an embodiment, when the electronic apparatus 100 determines that the user is having difficulty performing a movement during the user's exercise routine, the electronic apparatus 100 may display a video associated with the exercise currently being performed. As a result, the user may be able to smoothly perform the exercise routine based on the video associated with the exercise.

In addition, according to an embodiment, when the electronic apparatus 100 creates an exercise routine and provides information about the exercise routine, the electronic apparatus 100 may create and provide the user with images and videos regarding the exercise routine.

Further, according to an embodiment, the operation of providing an exercise routine may be performed only by the electronic apparatus 100, but this is only one embodiment, and the operation of providing an exercise routine may be performed by a plurality of apparatuses. For example, when the electronic apparatus 100 is a device without a display, such as a smart ring, the electronic apparatus 100 may perform the operation of obtaining the user's exercise performance ability, the external device 10 may perform the operations of updating the exercise routine based on the exercise performance ability and providing information about the exercise routine. Further, some of the operations of an embodiment of the present disclosure (e.g., updating the exercise routine based on the user's exercise performance ability) may also be performed by an external server.

Meanwhile, the methods according to an embodiment may be included and provided in a computer program product. The computer program product may be traded as a product between a seller and a purchaser. The computer program product may be distributed in the form of a storage medium (e.g., compact disc read only memory (CD-ROM)) that is readable by devices, may be distributed through an application store (e.g., PlayStore™) or directly between two user devices (e.g., smartphones), or may be distributed online (e.g., by downloading or uploading). In the case of an online distribution, at least part of the computer program product (e.g., a downloadable application) may be at least temporarily stored in a storage medium readable by a machine such as a server of the manufacturer, a server of an application store, or the memory of a relay server or may be temporarily generated.

The above-described various embodiments may be implemented as software including instructions stored in machine-readable storage media, which can be read by machine (e.g.: computer). The machine refers to a device that calls instructions stored in a storage medium, and can operate according to the called instructions, and the device may include an electronic apparatus (e.g.: TV) according to the aforementioned embodiments.

Meanwhile, the machine-readable storage media may be provided in a non-transitory storage medium. Here, ‘non-transitory storage medium’ merely means that the storage medium is tangible without including a signal (e.g. electromagnetic waves), and does not distinguish whether data are semi-permanently or temporarily stored in the storage medium. For example, the ‘non-transitory storage medium’ may include a buffer in which data is temporarily stored.

In case the instructions are executed by a processor, the processor may perform functions corresponding to the instructions by itself, or by using other components under its control. The instructions may include a code that is generated or executed by a compiler or an interpreter.

Although preferred embodiments of the present disclosure have been shown and described above, the disclosure is not limited to the specific embodiments described above, and various modifications may be made by one of ordinary skill in the art without departing from the gist of the disclosure as claimed in the claims, and such modifications are not to be understood in isolation from the technical ideas or prospect of the disclosure.

What is claimed is:

1. An electronic apparatus comprising:
a sensor;
a display;
memory storing instructions; and
at least one processor,
wherein the instructions, when executed by the at least one processor, cause the electronic apparatus to:
obtain data about a user;
obtain information about an exercise routine corresponding to the user based on the obtained data;
obtain information about an exercise performance ability of the user while the user is exercising using the exercise routine; and
update the exercise routine based on the exercise performance ability.

2. The apparatus of claim 1, wherein the instructions, when executed by the at least one processor, cause the electronic apparatus to:
provide query information to obtain information about at least one of the user's current condition or exercise ability; and
obtain data about the user according to a user input entered based on the query information.

3. The apparatus of claim 2, wherein the query information includes at least one of query information to inquire about the user's diet, sleeping, drinking alcohol, medication, activity amount, body fat, target exercise area, or satisfaction about an existing exercise routine.

4. The apparatus of claim 3, wherein the instructions, when executed by the at least one processor, cause the electronic apparatus to:
obtain information about whether the user is able to perform exercise and information about exercise intensity based on the obtained data;
identify an exercise goal based on the information about whether the user is able to perform exercise and information about exercise intensity;
analyze feedback information about the existing exercise routine; and
obtain information about an exercise routine corresponding to the user based on the exercise goal and the feedback information.

5. The apparatus as claimed in claim 4, wherein the instructions, when executed by the at least one processor, cause the electronic apparatus to:
based on the user drinking alcohol within a preset drinking period, taking a preset drug within a preset drug period, eating less than a threshold amount of food, sleeping less than a threshold sleeping time, or having a vitality score less than a preset vitality value, obtain information about an exercise routine of first intensity;
based on the user not drinking alcohol within the preset drinking period, not taking the preset drug within the preset drug period, eating more than the threshold amount of food, sleeping more than the threshold sleeping time, or having the vitality score more than the preset vitality value, obtain information about an increase in body fat;
based on the increase in body fat being less than a preset body fat value, obtain information about an exercise routine of second intensity; and
based on the increase in body fat being more than the preset body fat value, obtain information about an exercise routine of third intensity, wherein the first intensity is lower than the second intensity, and the second intensity is lower than the third intensity.

6. The apparatus of claim 3, wherein the instructions, when executed by the at least one processor, cause the electronic apparatus to:
calculate a condition score based on information about the user's meal amount, the user's last meal time, the user's sleep amount, whether the user drinks alcohol, the user's activity amount, the user's change in body fat, and a degree of performance of the existing exercise routine; and
obtain information about an exercise routine corresponding to the user by adjusting an exercise goal of the existing exercise routine based on the existing exercise routine and the condition score.

7. The apparatus of claim 1, wherein the instructions, when executed by the at least one processor, cause the electronic apparatus to:
obtain information about the user's condition and information about a progress of the exercise routine while the user is exercising using the exercise routine; and
update detailed settings of a next exercise included in the exercise routine based on the information about the user's condition and the information about the progress of the exercise routine.

8. The apparatus of claim 7, wherein the exercise routine is configured to include a plurality of exercises and a rest period between the plurality of exercises; and
wherein the instructions, when executed by the at least one processor, cause the electronic apparatus to provide information about the updated detailed settings of a next exercise during the rest period.

9. The apparatus of claim 8, wherein the detailed settings of the exercise are configured to include at least one of the number of progressions of exercise, a weight of equipment required for exercise, or the number of sets of exercise.

10. The apparatus of claim 1, wherein the exercise routine is configured to include a warm-up exercise, a main exercise, a rest period, and a cool-down exercise.

11. A controlling method of an electronic apparatus, the method comprising:
obtaining data, by the electronic apparatus, about a user;
obtaining information, by the electronic apparatus, about an exercise routine corresponding to the user based on the obtained data;
obtaining information, by the electronic apparatus, about an exercise performance ability of the user while the user is exercising using the exercise routine; and
updating the exercise routine, by the electronic apparatus, based on the exercise performance ability.

12. The method of claim 11, wherein the obtaining data about the user comprises:
providing query information to obtain information about at least one of the user's current condition or exercise ability; and
obtaining data about the user according to a user input entered based on the query information.

13. The method of claim 12, wherein the query information includes at least one of query information to inquire about the user's diet, sleeping, drinking, medication, activity amount, body fat, target exercise area, or satisfaction about an existing exercise routine.

14. The method of claim 13, wherein the obtaining information about an exercise routine comprises:
obtaining information about whether the user is able to perform exercise and information about exercise intensity based on the obtained data;
identifying an exercise goal based on the information about whether the user is able to perform exercise and information about exercise intensity;
analyzing feedback information about the existing exercise routine; and
obtaining information about an exercise routine corresponding to the user based on the exercise goal and the feedback information.

15. The method of claim 14, wherein the obtaining information about an exercise routine comprises:
based on the user drinking alcohol within a preset drinking period, taking a preset drug within a preset drug period, eating less than a threshold amount of food, sleeping less than a threshold sleeping time, or having a vitality score less than a preset vitality value, obtaining information about an exercise routine of first intensity;
based on the user not drinking alcohol within the preset drinking period, not taking the preset drug within the preset drug period, eating more than the threshold amount of food, sleeping more than the threshold sleeping time, or having the vitality score more than the preset vitality value, obtaining information about an increase in body fat;
based on the increase in body fat being less than a preset body fat value, obtaining information about an exercise routine of second intensity; and
based on the increase in body fat being more than the preset body fat value, obtaining information about an exercise routine of third intensity, wherein the first intensity is lower than the second intensity, and the second intensity is lower than the third intensity.

16. The method of claim 13, further comprising:
calculating a condition score based on information about the user's meal amount, the user's last meal time, the user's sleep amount, whether the user drinks alcohol, the user's activity amount, the user's change in body fat, and a degree of performance of the existing exercise routine; and
obtaining information about an exercise routine corresponding to the user by adjusting an exercise goal of the existing exercise routine based on the existing exercise routine and the condition score.

17. The method of claim 11, further comprising:
obtaining information about the user's condition and information about a progress of the exercise routine while the user is exercising using the exercise routine; and
updating detailed settings of a next exercise included in the exercise routine based on the information about the user's condition and the information about the progress of the exercise routine.

18. The method of claim 17, wherein the exercise routine is configured to include a plurality of exercises and a rest period between the plurality of exercises; and
wherein the instructions, when executed by the at least one processor, cause the electronic apparatus to provide information about the updated detailed settings of a next exercise during the rest period.

19. The method of claim 18, wherein the detailed settings of the exercise are configured to include at least one of the number of progressions of exercise, a weight of equipment required for exercise, or the number of sets of exercise.

20. A computer program product comprising a storage medium storing instructions configured to be executed by at least one processor of an electronic apparatus to perform a plurality of operations comprising:
obtaining data about a user;
obtaining information about an exercise routine corresponding to the user based on the obtained data;
obtaining information about an exercise performance ability of the user while the user is exercising using the exercise routine; and
updating the exercise routine based on the exercise performance ability.
